(19) United States
(12) Patent Application Publication
(10) Pub. No.: US 2020/0390665 A1
(43) Pub. Date: Dec. 17, 2020

(54) COLOURED AQUEOUS PARTICLE DISPERSION COMPRISING AT LEAST ONE PARTICULATE DYESTUFF, A MATT-EFFECT FILLER, FILM-FORMING POLYMER PARTICLES, A THICKENER AND AN IONIC POLYMERIC DISPERSANT

(71) Applicant: L'OREAL, Paris (FR)

(72) Inventors: Micheline EL ACHKAR, Chevilly Larue (FR); Carolona MARCAIS-HELIE, Chevilly Larue (FR)

(73) Assignee: L'OREAL, Paris (FR)

(21) Appl. No.: 16/954,238

(22) PCT Filed: Dec. 4, 2018
(86) PCT No.: PCT/EP2018/083478

§ 371 Date: Jun. 16, 2020

(30) Foreign Application Priority Data
Dec. 20, 2017 (FR) 1762598

(51) Int. Cl. A61K 8/04; A61K 8/29; A61K 8/25; A61K 8/81; A61K 8/89; A61K 8/90; A61Q 1/08; A61Q 1/10; A61Q 1/12

(52) U.S. Cl. CPC A61K 8/04; A61K 8/29; A61K 8/25; A61K 8/8147; A61K 8/8152; A61K 8/89; A61K 2800/594; A61Q 1/08; A61Q 1/10; A61Q 1/12; A61K 2800/10; A61K 2800/48; A61K 2800/43; A61K 8/90

(57) ABSTRACT

The present invention relates to a composition in the form of an aqueous particle dispersion, especially comprising a physiologically acceptable medium, especially for coating keratin materials, more particularly for making up and/or caring for keratin materials such as the skin, containing: a) an aqueous phase; and b) at least one matt-effect filler; and c) solid particles formed from at least one film-forming polymer suspended in said aqueous phase; and d) at least one ionic polymeric dispersant; and e) at least one thickener; and f) at least one particulate dyestuff. The invention also relates to a process for coating keratin materials, more particularly for making up and/or caring for keratin materials, such as the skin, characterized in that it comprises the application to the keratin materials of a composition as defined previously.

The present invention is directed towards proposing, for the field of caring for and/or making up keratin materials, especially the skin, a novel coloured aqueous particle dispersion that is most particularly advantageous with regard to its technical performance and the sensations it affords the user on application to said keratin materials and in particular to the skin.

Cosmetic compositions, for example foundations, are commonly used to give the skin an aesthetic colour, but also to enhance the beauty of irregular skin, by making it possible to hide marks and dyschromias, to reduce the visibility of relief imperfections such as pores and wrinkles, and to conceal spots, acne marks and scars. In this regard, the coverage and the matt effect or “soft focus” effect are the main properties desired.

Emulsions are generally appealing to consumers, in particular in the context of foundations, since they are easy to apply. For such compositions, it is sought to provide colour to enhance the beauty of the skin. Substantial coverage and a matt effect are also sought to hide skin defects and to make the complexion uniform. A high proportion of pigments is generally used for this purpose. It is also important for the pigments present in these compositions to have good dispersibility with a view to obtaining a stable and uniform composition. In these emulsions, consumers are also looking for products which, after application, afford a light texture with no thickness or heaviness effect and which do not transfer onto substrates in contact with the keratin materials such as the skin, for instance clothing. However, the emulsions generally used in makeup have a tendency to have poor performance as regards freshness, and to produce a greasy effect due to the large presence of oils in these emulsions. Furthermore, the light texture effect and/or the transfer-resistance effect are not always fully satisfactory.

There remains a need to find novel aqueous presentation forms based on pigments that can be used especially as foundations which have good dispersibility of the pigments so as to obtain a homogeneous composition, and which give good sensory properties in terms of freshness, a non-greasy effect, a light texture effect and a transfer-resistance effect, while at the same time having good makeup properties such as good colour coverage, a good matt effect and good persistence of these effects.

The Applicant has discovered, surprisingly, that this objective can be achieved with a composition in the form of an aqueous particle dispersion, especially comprising a physiologically acceptable medium, especially for coating keratin materials, more particularly for making up and/or caring for keratin materials, such as the skin, containing:

a) an aqueous phase; and
b) at least one matt-effect filler; and

c) solid particles formed from at least one film-forming polymer suspended in said aqueous phase; and

d) at least one ionic polymeric dispersant; and
e) at least one thickener; and
f) at least one particulate dyestuff.

This discovery forms the basis of the invention.

Thus, according to one of its aspects, the present invention relates to a composition in the form of an aqueous dispersion of solid particles, especially comprising a physiologically acceptable medium, especially for coating keratin materials, more particularly for making up and/or caring for keratin materials, such as the skin, containing:

a) an aqueous phase; and
b) at least one matt-effect filler; and
c) solid particles formed from at least one film-forming polymer suspended in said aqueous phase; and
d) at least one ionic polymeric dispersant; and
e) at least one thickener; and
f) at least one particulate dyestuff.

The invention also relates to a process for coating keratin materials, more particularly for making up and/or caring for keratin materials, such as the skin, characterized in that it comprises the application to the keratin materials of a composition as defined previously.

### Definitions

In the context of the present invention, the term “keratin material” especially means the skin (of the body, face, or around the eyes).

The term “physiologically acceptable” means compatible with the skin and/or its integuments, which has a pleasant colour, odour and feel, and which does not cause any unacceptable discomfort (stinging or tautness) liable to discourage the consumer from using this composition.

The term “aqueous particle dispersion” means any composition comprising an aqueous phase in which are dispersed particles that are insoluble in said aqueous phase; said composition not comprising any other phase that is immiscible in the aqueous phase of the composition, irrespective of the temperature at which the composition is manufactured.

The term “particle” denotes a solid material that is insoluble in the aqueous phase of the composition, irrespective of the temperature at which the composition is manufactured.

The term “fillers” should be understood as meaning colourless or white, mineral or synthetic particles of any shape, which are insoluble and dispersed in the aqueous phase of the composition, irrespective of the temperature at which the composition is manufactured.

### Matt-Effect or “Soft-Focus” Filler

The term matt-effect or “soft-focus” filler means a filler which gives the complexion greater transparency and a hazy effect. The matt-effect power of the compositions containing same may be characterized by means of the following protocol:

According to the invention, the term “matt-effect power” especially means that the composition produces a negative sheen value measured using a polarimetric camera, after application of a composition according to the invention to said keratin material.

The matt-effect fillers used in the compositions according to the present invention may be in lamellar (or platelet), spherical (or globular) form, in the form of fibres or in any other intermediate form between these defined forms.

In the present patent application, the term “spherical particles” means particles in the shape or substantially in the shape of a sphere, which are insoluble in the medium of the composition according to the invention, even at the melting point of the medium (approximately 100° C.).

The term “lamellar particles” means herein particles of parallelepipedal shape (rectangular or square surface), discoid shape (circular surface) or ellipsoid shape (oval surface), characterized by three dimensions: a length, a width and a height, which particles are insoluble in the medium of the composition according to the invention, even at the melting point of the medium (approximately 100° C.).

The matt-effect filler(s) used in the compositions according to the present invention are preferably present in concentrations ranging from 0.1% to 5% by weight and more preferentially from 1% to 3% by weight relative to the total weight of the composition.

According to a preferred embodiment, the filler(s) are chosen from a group comprising silica powders; silicone resin powders, especially polymethylsilsesquioxane powders; polytetrafluoroethylene powders; acrylic copolymer powders; hollow hemispherical silicone particles; hydrophobic silica aerogel particles; crosslinked elastomeric organopolysiloxane powders; crosslinked elastomeric organopolysiloxane powders coated with silicone resin; polyamide powders; starch powders; talc/TiO₂/alumina/silica composite powders; silica/TiO₂ composites; spherical cellulose particles; clays; perlite; boron nitride, and mixtures thereof.

By way of example, these matt-effect fillers may be chosen from:
silica powders such as porous silica microparticles, for instance Silica Beads SB150® and Silica Beads SB700® from Miyoshi, with a mean size of 5 μm, the Series-H® Sunspheres from Asahi Glass, for instance Sunsphere H33®, Sunsphere H51® and Sunsphere H53® with respective sizes of 3.5 and 5 μm; amorphous silica microspheres coated with polydimethylsiloxane, such as those sold under the name SA Sunsphere H-33® and SA Sunsphere H-53® sold by the company Asahi Glass; amorphous ellipsoid hollow silica particles sold under the name Silica Shells® by the company Kobo;
polytetrafluoroethylene (PTFE) powders, such as those sold under the name Ceridust 9205F® from Clariant;
silicone resin powders, especially polymethylsilsesquioxane powders such as those having the INCI name: Polymethylsilsesquioxane, such as the products sold under the name Tospearl 145A® by the company GE Silicone;
acrylic copolymer powders, especially polymethyl (meth)acrylate (PMMA) powders, for instance the PMMA particles Jurimer MBI® from Nihon Junyoki with a mean size of 8 μm, the hollow PMMA spheres sold under the name Covabeads LH85® by the company Sensient Cosmetic Technologies, the PMMA particles having the trade name Ganzpearl GMP08200 from the company Ganz Chemical; hollow styrene/acrylate copolymer spheres, such as those sold under the name Sunspheres Powder® from the company Röhm & Haas; the expanded vinylidene/acrylonitrile/methylene methacrylate microspheres sold under the name Expancel®; hollow spheres of acrylate/ethylhexyl acrylate crosspolymer copolymer, such as the products sold by the company Daito Kasei Kogyo under the name Maquibeads SP-10®; acrylate/ethylhexyl acrylate copolymer microspheres, such as those sold by the company Serisui Plastics under the name Techpolymer ACP-8C®;
hollow hemispherical silicone particles, of INCI name Methylsilanol/silicate crosspolymer, as described in applications JP-2003 128 788 and JP-A-2000-191789, for instance NLK 500®, NLK 506® and NLK 510® from Takemoto Oil and Fat;
hydrophobic silica aerogel particles (INCI name: Silica Silylate), such as the products sold by the company Dow Corning under the name Dow Corning VM-2270® Aerogel Fine Particles;
crosslinked elastomeric organopolysiloxane powders, such as Dow Corning 9701 Cosmetic Powder® from the company Dow Corning (INCI name: Dimethicone/vinyl dimethicone crosspolymer); or the products sold by the company Dow Corning under the name EP-9801 Hydrocosmetic Powder® (INCI name: Dimethicone/vinyl dimethicone crosspolymer (and) butylene glycol);
crosslinked elastomeric organopolysiloxane powders coated with silicone resin, especially with silsesquioxane resin, as described, for example, in patent U.S. Pat. No. 5,538,793. Such elastomer powders are available under the names KSP-100®, KSP-101®, KSP-102®, KSP-103®, KSP-104® and KSP-105® of INCI name: Vinyl dimethicone/methicone silsesquioxane crosspolymer and sold by the company Shin-Etsu; or the products having the INCI name: vinyl dimethicone/methicone silsesquioxane crosspolymer treated with PEG-7 glyceryl cocoate, polyquaternium-7 and methylsilanol tri-PEG-8 glyceryl cocoate sold by the company Miyoshi Kasei under the name MW-SRP-100®;
polyamide (Nylon®) powders, for instance the Nylon 12 particles of the Orgasol® type such as Orgasol 2002 EXD NAT COS® and Orgasol 2002 UD NAT COS® from the company Arkema;
starch powders, in particular aluminium starch octenylsuccinate, such as the product sold by the company Akzo Nobel under the name Dry Flo Plus®;
talc/TiO₂/alumina/silica composite powders, for instance the products sold under the name Coverleaf AR-80® by the company Catalyst & Chemicals;
silica/TiO₂ composites, such as those sold by the company Sunjin Chemical under the name Sunsil Tin 50;
clays, in particular talcs;
spherical cellulose particles, such as those sold by the company Daito Kasei under the name Cellulobeads USF®;
boron nitride;
perlite;
and mixtures thereof.

According to a particularly preferred form of the invention, among these matt-effect fillers, use will be made of those chosen from:
silica powders;
the polymethylsilsesquioxane powders of INCI name: Polymethylsilsesquioxane;
polymethyl (meth)acrylate (PMMA) powders;
perlite;
boron nitride;
and mixtures thereof.

According to a particularly preferred form of the invention, among these matt-effect fillers, use will be made of those chosen from boron nitride, polymethylsilsesquioxane powders, and mixtures thereof.

### Clays

Among the clays, mention may be made of clays of the smectite family, such as laponite, of the kaolinite family, such as kaolinite, dickite, nacrite, optionally modified clays of the halloysite, dombassite, antigorite, berthierine, pyrophyllite, montmorillonite, beidellite, vermiculite, talc, stevensite, hectorite, saponite, chlorite, sepiolite and illite family.

Clays are products that are already well known per se, which are described, for example, in the publication “Mineralogie des argiles” [“Clay Mineralogy”], S. Caillère, S. Hénin, M. Rautureau, 2nd Edition 1982, Masson, the teaching of which is included herein by way of reference.

Natural clay is a sedimentary rock composed to a large extent of specific minerals, silicates generally of aluminium. Kaolin is thus a natural clay.

The clays may also be synthetic. Thus, Sumecton mentioned below is a synthetic saponite.

The clays may also be chemically modified with various compounds, such as acrylic acids, polysaccharides (for example carboxymethylcellulose) or organic cations.

Preferably, in the context of the present invention, use is made of clays that are cosmetically compatible and acceptable with keratin materials. Clays that may especially be mentioned include kaolinite, montmorillonites, saponites, laponites, hectorites and illites. Mixtures of clays and natural clays may also be used.

Natural clays that may be mentioned include green clays, in particular rich in illite; clays rich in montmorillonite, known as fuller's earth, or such as bentonite or else white clays rich in kaolinite. As bentonites, mention may in particular be made of those sold under the names Bentone 38 VCG®, Bentone Gel CAO V®, Bentone 27 V® and Bentone Gel MIO V® by the company Elementis.

By way of clay rich in montmorillonite, mention may be made of the aluminium silicate hydrate sold under the name Gel White H® by the company Rockwood.

By way of saponite, which belongs to the montmorillonite family, mention may be made of synthetic saponite, in particular the product sold by the company Kunimine under the name Sumecton®.

According to a particular embodiment of the invention, the clay will be talc. The talc in accordance with the invention may be chosen more particularly from those sold under the names Rose Talc® and Talc SG-2000® sold by the company Nippon Talc, Luzenac Pharma M® sold by the company Luzenac, J-68BC® from US Corporation and Micro ACE-P-3® sold by the company Nippon Talc.

### Perlite

Perlite is a natural glass of volcanic origin, of glossy black or light-grey colour, resulting from the rapid cooling of lava, and which is in the form of small particles resembling pearls.

The perlites that may be used according to the invention are generally aluminosilicates of volcanic origin and have the composition:

70.0%-75.0% by weight of silica SiO₂
12.0%-15.0% by weight of aluminium oxide Al₂O₃
3.0%-5.0% of sodium oxide Na₂O
3.0%-5.0% of potassium oxide K₂O
0.5%-2% of iron oxide Fe₂O₃
0.2%-0.7% of magnesium oxide MgO
0.5%-1.5% of calcium oxide CaO
0.05%-0.15% of titanium oxide TiO₂.

Preferentially, the perlite particles used according to the invention will be in porous expanded form.

The perlite is ground, dried and then sized in a first step. The product obtained, known as perlite ore, is grey-coloured and has a size of about 100 μm. The perlite ore is subsequently expanded (1000° C./2 seconds) to give more or less white particles. When the temperature reaches 850-900° C., the water trapped in the structure of the material evaporates and brings about the expansion of the material, relative to its original volume. The expanded perlite particles in accordance with the invention may be obtained via the expansion process described in patent U.S. Pat. No. 5,002,698.

Preferably, the perlite particles used will be ground; in this case, they are known as Expanded Milled Perlite (EMP).

They preferably have a particle size defined by a median diameter D50 ranging from 0.5 to 50 μm and preferably from 0.5 to 40 μm.

Preferably, the perlite particles according to the invention have a particle size distribution such that at least 50% of the particles are less than 20 μm in size. In addition, they preferentially have a particle size distribution such that 90% by weight of the particles are less than 55 μm in size and preferably less than 40 μm in size. It is moreover preferred for 90% by weight of the particles to be greater than 5 μm in size.

Preferably, the perlite particles used have a loose bulk density at 25° C. ranging from 10 to 400 kg/m³ (standard DIN 53468) and preferably from 10 to 300 kg/m³.

The expanded perlite particles sold under the trade names Optimat 1430 OR® and Optimat 2550® by the company World Minerals, or the commercial products GK-110 Thin® and GK-110 Extra Thin® by the company Langfang Xindazhong Filter and the company Henan Zhongnan Filter Aid, will in particular be used.

### Boron Nitride

There are several polymorphic forms of boron nitride: hexagonal form boron nitrides (denoted h-BN), rhombohedral form boron nitrides (denoted r-BN), amorphous form boron nitrides (denoted a-BN), turbostratic boron nitrides (denoted t-BN), cubic form boron nitrides (denoted c-BN) and wurtzite-type hexagonal form boron nitrides (denoted w-BN).

Preferentially, the boron nitride particles in accordance with the invention are chosen from turbostratic boron nitride particles, i.e. particles whose crystallization planes may be slightly offset relative to the theoretical position of crystallization.

Turbostratic boron nitride is a precursor of hexagonal form born nitride (h-BN). It has the same type of characteristics and physical properties as exfoliated hexagonal boron nitride.

Preferentially, the boron nitride particles have an oxygen content ranging from 0.05% to 3% by weight, more preferentially from 0.1% to 2.5% by weight, relative to the total weight of the particle.

Preferentially, the boron nitride particles have a mean particle size ranging from 0.1 to 25 μm, preferably from 0.3 to 15 μm.

The particle size is measured according to a method of distribution by laser diffraction with an apparatus of the type Microtrac from Nikkiso or Mastersizer from Malvern, in particular by measuring the D[10], D[50] and D[90] values.

D[10] represents the maximum size that 10% by volume of the particles have.

D[50] represents the maximum size that 50% by volume of the particles have.

D[90] represents the maximum size that 90% by volume of the particles have.

The boron nitride particles can be modified with a surface-treatment agent making it possible to confer thereon amphiphilic properties and to promote the dispersibility thereof in the compositions comprising an oily phase and/or an aqueous phase.

Treatment agents that may be chosen include dimethylpolysiloxanes (dimethicone), linear siloxane polymers end-blocked with trimethoxysiloxy groups, polymethylhydrogenosiloxanes which are linear polysiloxanes called methicones, and polyoxyalkylenated polyalkylethersiloxanes such as the polymer PEG-8 methyl ether dimethicone.

The boron nitride particles in accordance with the invention may be chosen more particularly from the following commercial products:

UHP-1010® from Carborundum,
PUHP 1030L® from the company Saint Gobain Ceramics,
Boron Nitride Powder TRES BN PUHP 3002® from the company Saint Gobain Ceramics,
TRES BN PUHP 30005® from the company Saint Gobain Ceramics,
Leau3002® (INCI name: Boron Nitride (and) PEG-8 Methyl Ether Dimethicone) from the company Saint Gobain,
Ronaflair Boroneige SF-3 117774® from the company Merck,
Ronaflair Boroneige SQ-6® from MERCK, Softouch Boron Nitride CC6059® from the company Momentive,
Softtouch Boron Nitride CC 6097® from the company Momentive,
Softtouch COS102J® from the company Momentive.

### Film-Forming Polymer Particles

The composition according to the invention comprises solid particles formed from one or more film-forming polymers suspended in the aqueous phase of the composition.

Said solid film-forming polymer particles either may be used per se and are in suspension in the aqueous phase of the composition, or may be used in the form of particles in aqueous dispersion (latex or pseudolatex).

In the present patent application, the term “film-forming polymer” means a polymer that is capable of forming, by itself or in the presence of an auxiliary film-forming agent, a continuous film on a support, at a temperature ranging from 20° C. to 150° C.

Such a film-forming polymer present in the form of particles in aqueous dispersion is generally known as a (pseudo)latex, i.e. a latex or psuedolatex. Techniques for preparing these dispersions are well known to those skilled in the art.

The composition according to the invention may comprise one or more types of particle, these particles possibly varying as regards their size, their structure and/or their chemical nature.

The film-forming polymer(s) are preferentially present in a solids content ranging from 0.1% to 5% by weight, more preferentially ranging from 0.1% to 3% by weight, even more preferentially ranging from 0.1% to 2% by weight and more particularly from 0.1% to 1.5% by weight relative to the total weight of the composition.

These solid particles may be of anionic, cationic or neutral nature and may constitute a mixture of solid particles of different nature.

In the present invention, the term “aqueous” refers to a liquid medium based on water and/or hydrophilic solvents. This aqueous liquid medium may be constituted essentially of water. It may also comprise a mixture of water and of water-miscible organic solvent(s) (miscibility with water of greater than 50% by weight at 25° C.), for instance lower monoalcohols containing from 1 to 5 carbon atoms such as ethanol or isopropanol, glycols containing from 2 to 8 carbon atoms such as propylene glycol, ethylene glycol, 1,3-butylene glycol and dipropylene glycol, C₃-C₄ ketones and C₂-C₄ aldehydes.

Among the film-forming polymers that may be used in the composition of the present invention, mention may be made of synthetic polymers, of free-radical type or of polycondensate type, and polymers of natural origin, and mixtures thereof. In general, these polymers may be statistical polymers, block copolymers of A-B type, of A-B-A or else ABCD, etc. multiblock type, or even grafted polymers.

### Free-Radical Film-Forming Polymer

The term “free-radical polymer” means a polymer obtained by polymerization of unsaturated and especially ethylenic monomers, each monomer being capable of homopolymerizing (unlike polycondensates).

The film-forming polymers of free-radical type may in particular be acrylic and/or vinyl homopolymers or copolymers.

The vinyl film-forming polymers may result from the polymerization of ethylenically unsaturated monomers containing at least one acid group and/or esters of these acid monomers and/or amides of these acid monomers.

Ethylenically unsaturated monomers containing at least one acid group or monomer bearing an acid group that may be used include α,β-ethylenic unsaturated carboxylic acids such as acrylic acid, methacrylic acid, crotonic acid, maleic acid or itaconic acid. (Meth)acrylic acid and crotonic acid are used in particular, and more particularly (meth)acrylic acid.

The esters of acid monomers are advantageously chosen from (meth)acrylic acid esters (also known as (meth)acrylates), in particular (meth)acrylates of an alkyl, in particular of a C₁-C₂₀ and more particularly C₁-C₈ alkyl; (meth)acrylates of an aryl, in particular of a C₆-C₁₀ aryl; (meth)acrylates of a hydroxyalkyl, in particular of a C₂-C₆ hydroxyalkyl; and allyl methacrylate.

Among the alkyl (meth)acrylates that may be mentioned are methyl methacrylate, ethyl methacrylate, butyl methacrylate, isobutyl methacrylate, 2-ethylhexyl methacrylate and lauryl methacrylate.

Mention may be made, among hydroxyalkyl (meth)acrylates, of hydroxyethyl acrylate, 2-hydroxypropyl acrylate, hydroxyethyl methacrylate or 2-hydroxypropyl methacrylate.

Mention may be made, among aryl (meth)acrylates, of benzyl acrylate and phenyl acrylate.

The (meth)acrylic acid esters are in particular alkyl (meth)acrylates and allyl methacrylate.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl(meth)acrylamides, in particular of a C₂-C₁₂ alkyl. Among the N-alkyl(meth)acrylamides that may be mentioned are N-ethylacrylamide, N-t-butylacrylamide and N-t-octylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned above.

Examples of vinyl esters that may be mentioned are vinyl acetate, vinyl neodecanoate, vinyl pivalate, vinyl benzoate and vinyl t-butylbenzoate.

Styrene monomers that may be mentioned include styrene and α-methylstyrene. The list of monomers given is not limiting, and it is possible to use any monomer known to those skilled in the art included in the categories of acrylic and vinyl monomers (including monomers modified with a silicone chain).

Vinyl polymers that may also be used include silicone acrylic polymers.

Mention may also be made of polymers resulting from free-radical polymerization of one or more free-radical monomers inside and/or partially at the surface of pre-existing particles of at least one polymer chosen from the group constituted of polyurethanes, polyureas, polyesters, polyesteramides and/or alkyds. These polymers are generally referred to as “hybrid polymers”.

### Polycondensate

As film-forming polymer of polycondensate type, mention may be made of anionic, cationic, nonionic or amphoteric polyurethanes, acrylic polyurethanes, polyvinylpyrrolidone-polyurethanes, polyester-polyurethanes, polyether-polyurethanes, polyureas, polyurea/polyurethanes and silicone polyurethanes, and mixtures thereof.

The film-forming polyurethane may be, for example, an aliphatic, cycloaliphatic or aromatic polyurethane, polyurea/urethane or polyurea copolymer including, alone or as a mixture, at least one block chosen from:
a block of aliphatic and/or cycloaliphatic and/or aromatic polyester origin, and/or
a branched or unbranched silicone block, for example polydimethylsiloxane or polymethylphenylsiloxane, and/or
a block including fluoro groups.

The film-forming polyurethanes as defined in the invention may also be obtained from branched or unbranched polyesters or from alkyds including labile hydrogens, which are modified by reaction with a diisocyanate and a difunctional organic compound (for example dihydro, diamino or hydroxyamino), also including either a carboxylic acid or carboxylate group, or a sulfonic acid or sulfonate group, or alternatively a neutralizable tertiary amine group or a quaternary ammonium group.

Among the film-forming polycondensates, mention may also be made of polyesters, polyesteramides, fatty-chain polyesters, polyamides and epoxyester resins.

The polyesters may be obtained, in a known manner, by polycondensation of dicarboxylic acids with polyols, especially diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid, 2,6-naphthalenedicarboxylic acid. These dicarboxylic acid monomers may be used alone or as a combination of at least two dicarboxylic acid monomers. Among these monomers, the ones chosen in particular are phthalic acid, isophthalic acid and terephthalic acid.

The diol may be chosen from aliphatic, alicyclic and aromatic diols. The diol used is chosen in particular from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

Use may be made in the present invention of optionally modified polymers of natural origin, such as shellac resin, sandarac gum, dammar resins, elemi gums, copal resins, water-insoluble cellulose-based polymers such as nitrocellulose, modified cellulose esters especially including carboxyalkyl cellulose esters such as those described in patent application US 2003/185 774, and mixtures thereof.

According to a particular embodiment of the invention, said at least one film-forming polymer in the dispersed state is chosen from acrylic polymer dispersions, polyurethane dispersions, sulfopolyester dispersions, vinyl dispersions, polyvinyl acetate dispersions, vinylpyrrolidone, dimethylaminopropylmethacrylamide and lauryldimethylpropylmethacrylamidoammonium chloride terpolymer dispersions, dispersions of polyurethane/polyacrylic hybrid polymers and dispersions of particles of core-shell type, and mixtures thereof.

Various types of aqueous dispersions, in particular commercial aqueous dispersions, which are suited to the preparation of the composition in accordance with the present invention are detailed below.

1/ Thus, according to a preferred embodiment of the invention, the aqueous dispersion of polymer particles is an aqueous dispersion of acrylic polymer.

The acrylic polymer may be a styrene/acrylate copolymer and especially a polymer chosen from copolymers resulting from the polymerization of at least one styrene monomer and at least one C₁-C₁₈ alkyl (meth)acrylate monomer. As styrene monomers that may be used in the invention, examples that may be mentioned include styrene and α-methylstyrene, and in particular styrene.

The C₁-C₁₈ alkyl (meth)acrylate monomer is in particular a C₁-C₁₂ alkyl (meth)acrylate and more particularly a C₁-C₁₀ alkyl (meth)acrylate. The C₁-C₁₈ alkyl (meth)acrylate monomer may be chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate.

As acrylic polymer in aqueous dispersion, use may be made according to the invention of the styrene/acrylate copolymer sold under the name Joncryl SOX-8211® by the company BASF, the acrylic polymer sold under the reference Acronal® DS-6250 by the company BASF, or the acrylic copolymer Joncryl® 95 by the company BASF.

As acrylic polymer in aqueous dispersion, use will be made more particularly according to the invention of an aqueous dispersion of aminomethylpropanol salt of a copolymer of allyl methacrylate and of one or more monomers chosen from acrylic acid, methacrylic acid or an ester thereof of INCI name: AMP acrylates/allyl methacrylate copolymer, for instance the commercial product Fixate G-100L PR Polymer® in the form of an aqueous dispersion at 25-28% by weight by the company Lubrizol Advanced Materials, Inc.

2/ According to one embodiment variant of the invention, the aqueous dispersion of polymer particles is an aqueous dispersion of polyester-polyurethane and/or polyether-polyurethane particles, in particular in anionic form.

The anionic nature of the polyester-polyurethanes and of the polyether-polyurethanes used according to the invention is due to the presence in their constituent units of groups bearing a carboxylic acid or sulfonic acid function.

The polyester-polyurethane or polyether-polyurethane particles used according to the invention are generally sold in aqueous dispersion form.

The particle content of said dispersions currently available on the market ranges from about 20% to about 50% by weight relative to the total weight of the dispersion.

Among the anionic polyester-polyurethane dispersions that may be used in the aqueous varnishes according to the invention, mention may be made in particular of the product sold under the name Avalure UR 405® by the company Noveon.

Among the anionic polyether-polyurethane particle dispersions that may be used according to the invention, mention may be made in particular of the products sold under the name Avalure UR 450® by the company Noveon and under the name Neorez R 970® by the company DSM.

According to a particular embodiment of the invention, use may be made of a mixture of commercial dispersions constituted of anionic polyester-polyurethane particles as defined above and of anionic polyether-polyurethane particles also defined above.

For example, use may be made of a mixture constituted of the dispersion sold under the name Sancure 861® or a mixture of the product sold under the name Avalure UR 405® and of the product sold under the name Avalure UR 450®, these dispersions being sold by the company Noveon.

3/ According to another particular embodiment of the invention, the aqueous dispersion used comprises a mixture of at least two film-forming polymers in the form of solid particles that differ by their respective glass transition temperatures (Tg).

In particular, according to one embodiment of the invention, the composition in accordance with the invention may comprise at least one first film-forming polymer in the dispersed state and at least one second film-forming polymer in the dispersed state, said first and second polymers having different Tg values and, preferably, the Tg of the first polymer (Tg₁) is higher than the Tg of the second polymer (Tg₂). In particular, the difference between the Tg₁ and Tg₂ values is, as an absolute value, at least 10° C. and preferably at least 20° C.

More precisely, it comprises in an acceptable aqueous medium:
solid particles dispersed in the aqueous medium of a first film-forming polymer having at least one glass transition temperature Tg₁ greater than or equal to 20° C., and
solid particles dispersed in the aqueous medium of a second film-forming polymer having at least one glass transition temperature Tg₂ less than or equal to 70° C.

This dispersion generally results from a mixing of two aqueous dispersions of film-forming polymer.

The first film-forming polymer has at least one, and in particular has one, glass transition temperature Tg₁ greater than or equal to 20° C., in particular ranging from 20° C. to 150° C. and advantageously greater than or equal to 40° C., in particular ranging from 40° C. to 150° C. and in particular greater than or equal to 50° C., in particular ranging from 50° C. to 150° C.

The second film-forming polymer has at least one, and in particular has one, glass transition temperature Tg₂ less than or equal to 70° C., in particular ranging from −120° C. to +70° C., in particular less than 50° C., in particular ranging from −60° C. to +50° C. and more particularly ranging from −30° C. to +30° C.

The measurement of the glass transition temperature (Tg) of a polymer is performed by DMTA (dynamic and mechanical temperature analysis) as described below.

To measure the glass transition temperature (Tg) of a polymer, viscoelasticity tests are performed with a Polymer Laboratories DMTA machine, on a sample of film. This film is prepared by pouring the aqueous dispersion of film-forming polymer in a Teflon-coated matrix followed by drying at 120° C. for 24 hours. A film is then obtained, from which specimens are cut out (for example using a punch). These specimens are typically about 150 μm thick, from 5 to 10 mm wide and have a useful length of about 10 to 15 mm. A tensile stress is imposed on this sample. The sample undergoes a static force of 0.01 N on which is superimposed a sinusoidal displacement of ±8 μm at a frequency of 1 Hz. The test is thus performed in the linear range, at low levels of deformation. This tensile stress is applied to the sample at temperatures ranging from −150° C. to +200° C., with a temperature variation of 3° C. per minute.

The complex modulus E*=E′+iE″ of the polymer tested is then measured as a function of the temperature.

From these measurements, the dynamic moduli E′ and E″ and the damping power: tgδ=E″/E′ are deduced.

The curve of the tgδ values is then plotted as a function of the temperature; this curve presents at least one peak. The glass transition temperature Tg of the polymer corresponds to the temperature at the top of this peak.

When the curve has at least two peaks (in this case, the polymer has at least two Tg values), the value taken as the Tg of the polymer tested is the temperature for which the curve has a peak of the largest amplitude (i.e. corresponding to the largest tgδ value; in this case, only the “major” Tg is considered as the Tg value of the polymer tested).

In the present invention, the transition temperature Tg₁ corresponds to the “major” Tg (in the predefined sense) of the first film-forming polymer when it has at least two Tg values; the glass transition temperature Tg₂ corresponds to the “major” Tg of the second film-forming polymer when it has at least two Tg values.

The first film-forming polymer and the second film-forming polymer may be chosen, independently of each other, from free-radical polymers, polycondensates and polymers of natural origin as defined previously having the glass transition temperature characteristics defined previously.

As first film-forming polymer in aqueous dispersion, use may be made of the aqueous polymer dispersions sold under the names Neorez R-989® by the company DSM, Joncryl 95® and Joncryl 8211® by the company BASF.

As second film-forming polymer in aqueous dispersion, use may be made, for example, of the aqueous polymer dispersions sold under the names Avalure UR-405® and Avalure UR-460® by the company Noveon or Acrilem IC89RT® by the company ICAP, and Neocryl A-45® by the company DSM.

The film-forming polymer of the aqueous dispersion Avalure UR-460® is a polyurethane obtained by polycondensation of polytetramethylene oxide, tetramethylxylylene diisocyanate, isophorone diisocyanate and dimethylolpropionic acid.

According to one embodiment of the invention, use is made, as first and second film-forming polymers in aqueous dispersion, of the combination of styrene/acrylate polymer dispersion such as the dispersion sold under the reference Joncryl 8211® by BASF and of acrylic polymer dispersion such as the dispersion sold under the reference Neocryl A-45® by DSM.

According to another embodiment of this particular embodiment of point 3/above of the invention, use is made, as first film-forming polymer in aqueous dispersion, of an acrylic polymer dispersion such as the dispersion sold under the reference Joncryl 95® by BASF and, as second film-forming polymer, of a dispersion of anionic polyurethane polymer sold under the reference Avalure UR405® by DSM.

4/ According to yet another embodiment of the invention, the composition comprises at least one particular multi-phase aqueous dispersion.

More specifically, it comprises, in an acceptable aqueous medium, a particle dispersion, the particles comprising at least one at least partly external flexible phase including at least one flexible polymer having at least one glass transition temperature Tg₂ and at least one at least partly internal rigid phase, the rigid phase being an amorphous material having at least one glass transition temperature Tg₁, Tg₁ being higher than Tg₂, the flexible polymer being at least partially attached by chemical grafting to the rigid phase.

In particular, the difference between the Tg₁ and Tg₂ values is, as an absolute value, at least 10° C. and preferably at least 20° C.

The particles according to the invention, also referred to as multi-phase (or composite) particles, are particles comprising at least one flexible phase and at least one rigid phase.

The flexible polymer of the particles in dispersion may have at least one glass transition temperature of less than or equal to 70° C., especially ranging from −120° C. to 70° C., more particularly less than or equal to 30° C., especially ranging from −60° C. to 30° C.

The flexible polymer may be chosen from block and/or random polymers. The term “block and/or random polymers” means polymers in which the distribution of the monomers on the main chain or the side chain units is in block and/or random form.

The flexible polymer may be chosen from radical polymers, polycondensates and silicone polymers. The flexible polymer may be chosen from polyacrylics, polymethacrylics, polyamides, polyurethanes, polyolefins, especially polyisoprenes, polybutadienes, polyisobutylenes (PIB), polyesters, polyvinyl ethers, polyvinyl thioethers, polyoxides, polysiloxanes and especially polydimethylsiloxanes (PDMS), and combinations thereof. The term “combinations” means copolymers which may be formed from the monomers leading to the formation of said polymers.

In particular, the flexible polymer may be chosen from poly(meth)acrylics, polyurethanes, polyolefins and polysiloxanes.

The amorphous material of the rigid phase has a glass transition temperature of greater than 20° C., especially ranging from 20° C. to 150° C., in particular greater than or equal to 30° C., especially ranging from 30° C. to 150° C., preferably greater than or equal to 40° C., more particularly ranging from 40° C. to 150° C., or even greater than or equal to 50° C., especially ranging from 50° C. to 150° C.

The amorphous material of the rigid phase may be a polymer, especially a block and/or random polymer. It may be a polymer chosen from polyacrylics, polymethacrylics, for instance poly(meth)acrylic acid, poly(meth)acrylamides, polyvinyls, polyvinyl esters, polyolefins, polystyrenes, polyvinyl halides such as polyvinyl chloride (PVC), polyvinyl nitriles, polyurethanes, polyesters, polyamides, polycarbonates, polysulfones, polysulfonamides, polycyclics bearing a carbon ring in the main chain, for instance polyphenylenes or polyoxyphenylenes, and combinations thereof.

Advantageously, the amorphous material of the rigid phase may be a polymer chosen from polyacrylics, polymethacrylics, for instance poly(meth)acrylic acid, poly(meth)acrylamides, polyvinyls, polyvinyl esters, polyolefins, polystyrenes, polyvinyl halides such as polyvinyl chloride (PVC), polyvinyl nitriles, polyurethanes, polyamides and polyesters.

According to a particular embodiment of the invention, the flexible and rigid phases of the multi-phase particles may comprise at least one radical polymer obtained via, or even essentially via, polymerization of monomers chosen from the group formed by:
(meth)acrylic acid esters, for instance alkyl (meth)acrylates, especially bearing a C₁-C₈ alkyl group,
vinyl esters of linear or branched carboxylic acids, such as vinyl acetate or vinyl stearate,
styrene and derivatives thereof, such as chloromethylstyrene and alpha-methylstyrene,
conjugated dienes, such as butadiene or isoprene,
acrylamide, methacrylamide and acrylonitrile,
vinyl chloride, and
(meth)acrylic acid.

The selection of monomers (nature and content), which may be a single monomer or a mixture of at least two monomers, of the flexible polymer and of the amorphous material of the rigid phase is determined by the glass transition temperature that it is desired to give to each polymer.

The polymers of the rigid and/or flexible phases may be crosslinked with the aid of monomers bearing at least two copolymerizable double bonds, chosen, for example, from:
conjugated dienes, such as butadiene or isoprene,
allylic esters of alpha,beta-unsaturated carboxylic acids such as allyl acrylate or allyl methacrylate,
allylic esters of alpha,beta-unsaturated dicarboxylic acids such as diallyl maleate,
polyacrylics or polymethacrylics generally comprising at least two ethylenic unsaturations, such as ethylene glycol dimethacrylate, 1,3-butylene glycol dimethacrylate, 1,4-butanediol diacrylate or pentaerythrityl tetraacrylate;
polyvinyls such as divinylbenzene or trivinylbenzene, and
polyallylics such as triallyl cyanurate.

Chemical grafting makes it possible, via the formation of covalent bonds, to stably bond the rigid phase and the flexible phase of the multi-phase particles.

The chemical grafting may be performed by block radical polymerization (also known as sequential polymerization) according to the procedures that are well known to those skilled in the art. Block polymerization consists in polymerizing, in a first step, the monomers of the rigid polymer (polymer forming the rigid phase of the particles) and then continuing the polymerization in a second step with the monomers forming the flexible polymer (polymer forming the flexible phase of the particles). In this way, the polymer chains of the flexible phase are at least partly bonded via covalent bonding to the chains of the polymer of the rigid phase, the covalent bonding resulting from the polymerization of a monomer of the flexible polymer with a monomer of the rigid polymer.

Advantageously, the monomers of the polymer of the outer flexible phase have greater affinity for the dispersing medium than the monomers of the polymer of the inner rigid phase.

The flexible polymer may be grafted onto the rigid polymer via a grafting monomer, said grafting monomer possibly being a monomer bearing several double bonds (ethylenic bonds), in particular a monomer bearing two ethylenic double bonds. The grafting monomer may be a conjugated diene such as those described previously or an allylic ester (especially diester) of alpha,beta-unsaturated dicarboxylic acids such as those described previously (for instance diallyl maleate) which bear two polymerizable functions (ethylenic double bond) of different reactivities: one of the polymerizable functions (ethylenic double bond) of the grafting monomer is polymerized with the polymer of the amorphous material of the rigid phase (rigid polymer) and the other polymerizable function (ethylenic double bond) of the same grafting monomer is polymerized with the flexible polymer.

When the flexible polymer or the polymer of the rigid phase is a polycondensate, a polycondensate bearing at least one ethylenic unsaturation that is capable of reacting with a monomer also including an ethylenic unsaturation to form a covalent bond with the polycondensate is used in particular. Polycondensates comprising one or more ethylenic unsaturations are obtained especially via polycondensation of monomers such as allyl alcohol, vinylamine or fumaric acid. It is possible, for example, to polymerize vinyl monomers with a polyurethane bearing vinyl groups in or at the end of the polyurethane chain and thus to graft a vinyl polymer onto a polyurethane; a dispersion of particles of such a grafted polymer is especially described in the publications The structure and properties of acrylic-polyurethane hybrid emulsions, Hiroze M., Progress in Organic Coatings, 38 (2000), pages 27-34; Survey of the applications, properties, and technology of crosslinking emulsions, Bufkin B., Journal of Coatings Technology, vol. 50, No. 647, December 1978.

The same grafting principle applies for silicones using silicones including vinyl groups making it possible to polymerize vinyl monomers on the silicone and thus to graft vinyl polymer chains onto a silicone.

The particles bearing rigid and flexible phases generally have a size ranging from 1 nm to 1 μm and in particular ranging from 10 nm to 1 μm.

The flexible phase may be present in the particles in a content of at least 1% by volume, relative to the total volume of the particle, especially of at least 5% by volume, in particular of at least 10% by volume and more particularly of at least 25% by volume. The flexible phase may be present in the particles in a content ranging up to 99.999% by volume, especially up to 99.9% by volume, in particular up to 99% by volume and more particularly up to 95% by volume. In particular, the flexible phase may be present in the particles in a content ranging from 1% to 99.999% by volume, in particular ranging from 5% to 99.9% by volume, more particularly ranging from 10% to 99.9% by volume, especially ranging from 25% to 99.9% by volume, more particularly ranging from 50% to 95% by volume, or even ranging from 50% to 90% by volume.

In all cases, the rigid phase and the flexible phase are incompatible, i.e. they may be distinguished by using techniques that are well known to those skilled in the art, for instance the technique of observation by electron microscopy or measurement of the glass transition temperatures of the particles by differential calorimetry. The multi-phase particles are thus non-homogeneous particles.

The morphology of the flexible and rigid phases of the dispersed particles may be, for example, of core-shell type, with shell particles completely surrounding the core, but also of core-shell type with a multiplicity of cores, or an interpenetrating network of phases. In the multi-phase particles, the flexible phase is at least partly and in particular predominantly external, and the rigid phase is at least partly and in particularly predominantly internal.

The multi-phase particles may be prepared via consecutive series of polymerization, with different types of monomers. The particles of a first family of monomers are generally prepared in a separate step, or formed in situ by polymerization. Next, or at the same time, at least one other family of other monomers is polymerized in at least one additional polymerization step. The particles thus formed have at least one at least partly internal structure, or core structure, and at least one at least partly external structure, or shell structure. The formation of a “multi-layer” heterogeneous structure is thus possible. A wide variety of morphologies may result therefrom, of the core-shell type, but also, for example, with fragmented inclusions of the rigid phase in the flexible phase. According to the invention, it is essential for the at least partly external flexible phase structure to be more flexible than the at least partly internal rigid phase structure.

According to a particular embodiment of the invention, the multi-phase particles may be dispersed in an aqueous medium, especially a hydrophilic medium. The aqueous medium may be predominantly constituted of water, and in particular virtually totally of water. These dispersed particles thus form an aqueous polymer dispersion, which is generally known as a latex or pseudolatex. The term “latex” means an aqueous dispersion of polymer particles as may be obtained by emulsion polymerization of at least one monomer.

The multi-phase particle dispersion is generally prepared via at least one emulsion polymerization, in an essentially aqueous continuous phase, using reaction initiators such as photochemical or thermal initiators for a radical polymerization, optionally in the presence of additives such as stabilizers, chain-transfer agents, and/or catalysts.

As solid particles of film-forming polymer according to the invention, use may be made more preferentially of:
aqueous dispersions of acrylic polymer, such as those sold under the names Acronal DS-6250® by the company BASF, Neocryl A-45®, Neocryl XK-90®, Neocryl A-1070®, Neocryl A-1090®, Neocryl BT-62®, Neocryl A-1079® and Neocryl A-523® by the company DSM, Joncryl 95® and Joncryl 8211® by the company BASF, Daitosol 5000 AD® (INCI name: Acrylates Copolymer) or Daitosol 5000 SJ® (INCI name: Acrylates/ethylhexyl acrylate copolymer) by the company Daito Kasei Kogyo; Syntran 5760® (INCI name: Styrene/acrylates/ammonium methacrylate copolymer) by the company Interpolymer, aqueous dispersions of aminomethylpropanol salt of a copolymer of allyl methacrylate and of one or more monomers chosen from acrylic acid, methacrylic acid or an ester thereof of INCI name: AMP acrylates/allyl methacrylate copolymer, for instance the commercial product Fixate G-100L PR Polymer® in the form of an aqueous dispersion at 25-28% by weight by the company Lubrizol Advanced Materials, Inc.,
the aqueous polyurethane dispersions sold under the names Neorez R-981® and Neorez R-974® by the company DSM, Avalure UR-405®, Avalure UR-410®, Avalure UR-425®, Avalure UR-450®, Sancure 875®, Avalure UR 445® and Avalure UR 450® by the company Noveon, and Impranil 85® by the company Bayer,
sulfopolyester dispersions, such as those sold under the brand name Eastman AQ® by the company Eastman Chemical Products,
vinyl dispersions such as Mexomere PAM®, aqueous dispersions of polyvinyl acetate such as Vinybran® from the company Nisshin Chemical or the products sold by the company Union Carbide, aqueous dispersions of vinylpyrrolidone, dimethylaminopropylmethacrylamide and lauryldimethylpropylmethacrylamidoammonium chloride terpolymer such as Styleze W® from ISP,
aqueous dispersions of polyurethane/polyacrylic hybrid polymer such as the products sold under the references Hybridur® by the company Air Products or Duromer® from National Starch,
aqueous dispersions of particles of core-shell type such as the products sold by the company Arkema under the reference Kynar® (core: fluorinated—shell: acrylic) or alternatively those described in U.S. Pat. No. 5,188,899 (core: silica—shell: silicone) and mixtures thereof.

According to a particularly preferred form of the invention, use will be made of an aqueous dispersion of aminomethylpropanol salt of a copolymer of allyl methacrylate and of one or more monomers chosen from acrylic acid, methacrylic acid or an ester thereof of INCI name: AMP acrylates/allyl methacrylate copolymer, for instance the commercial product Fixate G-100L PR Polymer® in the form of an aqueous dispersion at 25-28% by weight by the company Lubrizol Advanced Materials, Inc.).

### Ionic Polymeric Dispersant

The ionic polymeric dispersants are preferably chosen from:
(meth)acrylic acid homopolymers and salts thereof,
(meth)acrylic acid copolymers and salts thereof,
styrene/(meth)acrylic acid copolymers and salts thereof,
vinylnaphthalene/(meth)acrylic acid copolymers and salts thereof,
styrene/maleic acid copolymers and salts thereof,
vinylnaphthalene/maleic acid copolymers and salts thereof,
maleic anhydride copolymers and salts thereof,
mixtures thereof.

The term “salts” means the salts of an alkali metal such as Na, Li or K of said acids, the salts of monoethanolamine, diethanolamine or triethanolamine or basic amino acids such as lysine or arginine of said acids, and mixtures thereof.

Among the maleic anhydride copolymers and salts thereof, mention may be made in particular of the copolymers obtained by copolymerization of one or more maleic anhydride comonomers and of one or more comonomers chosen from vinyl acetate, vinyl alcohol, vinylpyrrolidone, olefins including from 2 to 20 carbon atoms, for instance octadecene, ethylene, isobutylene, diisobutylene, isooctylene, and styrene, and mixtures thereof, the maleic anhydride comonomers which may optionally partially or totally hydrolysed, and more particularly copolymers of maleic anhydride and of diisobutylene and salts thereof, and more particularly the product sold under the name Orotan 731 DP® by the company Rhodia.

According to a particular embodiment of the invention, the dispersant(s) thus correspond to formula (I) below:

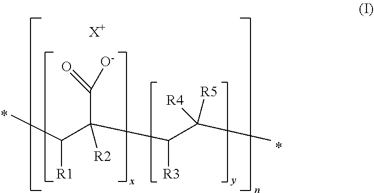

in which:
R1 represents a hydrogen atom, a methyl group or a group
*—C(O)O⁻/X⁺ or a group *—C(O)O—R6, with R6 representing a linear or branched C₁-C₅₀, preferably C₁-C₈, preferably C₁-C₄ alkyl group,
R2, R3 and R4, which may be identical or different, representing a hydrogen atom or a methyl group,
R5 representing a phenyl group or a linear or branched C₁-C₅₀, preferably C₁-C₂₀ alkyl group, or a group *—C(O)O—R7, with R7 representing a linear or branched C₁-C₅₀, preferably C₁-C₈, preferably C₁-C₄ alkyl group,
x represents an integer ranging from 1 to 100,
y represents an integer ranging from 0 to 100,
n representing an integer between 1 and 1000,
x, y and n being chosen such that the equation “n (x+y)” is greater than or equal to 20 and preferably greater than or equal to 50,
X⁺ representing a cation chosen especially from a sodium atom, a potassium atom and an ammonium group, and preferably being an ammonium group.

According to a more particular embodiment, the dispersant(s) thus correspond to formula (I′) below:

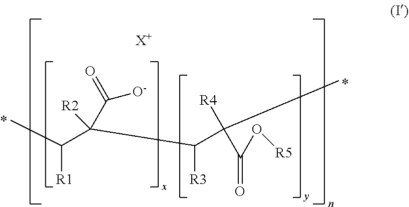

in which:
R1, R2, R3 and R4 represent a hydrogen atom or a C₁-C₆ alkyl group, in particular a methyl,
R5 representing a linear or branched C₁-C₅₀, preferably C₁-C₈, preferably C₁-C₄ alkyl group, in particular a methyl,
x represents an integer ranging from 1 to 100,
y represents an integer ranging from 0 to 100,
n representing an integer between 1 and 1000,
x, y and n being chosen such that the equation “n (x+y)” is greater than or equal to 20 and preferably greater than or equal to 50,
X⁺ representing a cation chosen especially from a sodium atom, a potassium atom and an ammonium group, and preferably being an ammonium group.

According to a more particular embodiment, the dispersant(s) thus correspond to formula (I″) below:

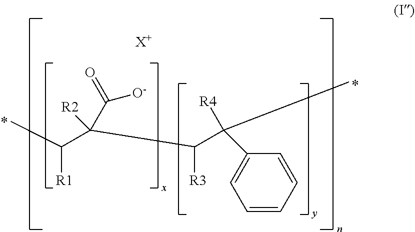

in which:
R1 represents a group *—C(O)O⁻/X⁺ or a group *—C(O)O—R5, with R5 representing a linear or branched C₁-C₅₀, preferably C₁-C₈, preferably C₁-C₄ alkyl group, in particular a methyl,
R2, R3 and R4 representing a hydrogen atom or a C₁-C₆ alkyl group, in particular a methyl,
x represents an integer ranging from 1 to 100,
y represents an integer ranging from 0 to 100,
n representing an integer between 1 and 1000,
x, y and n being chosen such that the equation “n (x+y)” is greater than or equal to 20 and preferably greater than or equal to 50,
X⁺ representing a cation chosen especially from a sodium atom, a potassium atom and an ammonium group, and preferably being an ammonium group.

Preferably, a composition in accordance with the invention comprises at least one ionic polymeric dispersant chosen from:
salts of copolymers of at least two or more monomers chosen from the group comprising acrylic acid, methacrylic acid or an ester thereof,
salts of styrene/maleic anhydride copolymer,
salts of methacrylic acid homopolymer,
and mixtures thereof.

Examples that may be mentioned include the following polymeric dispersants of ionic polymer type:
ammonium salts of a copolymer of at least two or more monomers chosen from the group comprising acrylic acid, methacrylic acid or an ester thereof, of INCI name: Ammonium acrylates copolymer, sold, for example, under the name Syntran KLG219-CG® by the company Interpolymer or Dispex AA 4040® from the company BASF,
ammonium salts of styrene/maleic anhydride (50/50) copolymer, especially in aqueous solution at 30%, sold under the reference SMA 1000H® by the company Arkema,
sodium salts of styrene/maleic anhydride copolymer, especially in aqueous solution at 40%, sold under the reference SMA 1000HNa® by the company Arkema,
a homopolymer of the sodium salt of methacrylic acid of INCI name: Sodium polymethacrylate, especially as an aqueous 25% solution sold under the reference Darvan® 7-N by the company R.T. Vanderbilt Company, Inc.,
mixtures thereof.

Use will be made more particularly of a polymer of the sodium salt of methacrylic acid of INCI name: Sodium polymethacrylate, especially as an aqueous 25% solution sold under the reference Darvan® 7-N by the company R.T. Vanderbilt Company, Inc.

A composition according to the invention preferably includes a total content of ionic polymeric dispersant(s) of greater than or equal to 0.05% by weight and less than or equal to 3% by weight, relative to the total weight of the composition, in particular ranging from 0.1% to 2% by weight, relative to the total weight of the composition, more particularly ranging from 0.1% to 1.5% by weight and even more particularly ranging from 0.1% to 1% by weight, relative to the total weight of the composition.

### Aqueous Phase

The composition according to the invention comprises an aqueous phase.

The term “aqueous phase” means a phase comprising water and also all the water-soluble or water-miscible solvents and ingredients.

The aqueous phase may contain a demineralized water or alternatively a floral water such as cornflower water and/or a mineral water such as Vittel water, Lucas water or La Roche Posay water and/or a spring water.

According to a preferential embodiment, the aqueous phase is present in a content ranging from 70% to 95% by weight relative to the total composition, preferably in a content ranging from 80% to 90% by weight relative to the total weight of the composition.

According to a preferential embodiment, water is present in a content ranging from 40% to 80% by weight relative to the total composition, preferably in a content ranging from 60% to 70% by weight relative to the total weight of the composition.

### Thickener

For the purposes of the present invention, the term “thickener” means any organic or inorganic compound that is capable of increasing the viscosity of the aqueous composition according to the invention.

The thickeners in accordance with the invention are preferably water-soluble.

For the purposes of the invention, the term “water-soluble thickener” means any thickener that can be fully dissolved in molecular form or that is miscible in a liquid aqueous phase or that can be dissolved in colloidal form (for example in micellar form) in a liquid aqueous phase.

As thickeners that may be used in the compositions according to the invention, mention may be made of:
polyacrylic acids of the type such as Synthalen K®;
polyacrylic acids crosslinked with a pentaerythrityl allyl ether, a sucrose allyl ether or a propylene allyl ether of Carbomer type;
copolymers of at least two monomers chosen from acrylic acid, methacrylic acid or an ester thereof of INCI name Acrylates copolymer, in particular the product sold under the name Carbopol Aqua SF1® by the company Lubrizol Advanced Materials, Inc.,
copolymers of acrylic acid and of acrylamide, sold in the form of their sodium salt under the names Reten® by the company Hercules;
crosslinked polyacrylic acid/C10-C30 alkyl acrylate copolymers of INCI name: Acrylates/C10-C30 alkyl acrylate crosspolymer, sold under the trade name Pemulen® or Carbopol 1382® sold by the company Lubrizol Advanced Materials, Inc.,
an acrylamidomethylpropanesulfonic acid polymer partially neutralized with ammonia and crosslinked, of INCI name: Ammonium polyacryloyldimethyl taurate sold under the trade name Hostacerin AMPS® by the company Clariant,
AMPS®/acrylamide copolymers of Sepigel® or Simulgel® type sold by the company SEPPIC, and
AMPS®/polyoxyethylenated alkyl methacrylate copolymers (crosslinked or non-crosslinked), and mixtures thereof.

Other examples of thickening polymers that may be mentioned include:
proteins, for instance proteins of plant origin such as wheat or soybean proteins; proteins of animal origin such as keratins, for example keratin hydrolysates and sulfonic keratins;
anionic, cationic, amphoteric or nonionic chitin or chitosan polymers;
cellulose polymers such as hydroxyethylcellulose, hydroxypropylcellulose, methylcellulose, ethylhydroxyethylcellulose and carboxymethylcellulose, and also quaternized cellulose derivatives;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;
associative polyurethanes such as the C16-OE120-C16® polymer from the company Servo Delden (sold under the name SER AD FX1100®, this molecule bearing a urethane function and having a weight-average molecular weight of 1300), OE being an oxyethylene unit, Rheolate 205® bearing a urea function, sold by the company Rheox, or Rheolate 208® or 204® (these polymers being sold in pure form) or DW 1206B® from Röhm & Haas bearing a C₂₀ alkyl chain and a urethane bond, sold at 20% solids in water. It is also possible to use solutions or dispersions of these associative polyurethanes, especially in water or in aqueous-alcoholic medium. Examples of such polymers that may be mentioned include SER AD FX1010, SER AD FX1035® and SER AD FX1070® from the company Servo Delden, and Rheolate 255®, Rheolate 278® and Rheolate 244® sold by the company Rheox. It is also possible to use the products DW 1206F® and DW 1206J®, and also Acrysol RM 184® or Acrysol 44® from the company Röhm & Haas, or alternatively Borchigel LW 44® from the company Borchers;
optionally modified polymers of natural origin, such as:
gum arabic, guar gum, xanthan derivatives, karaya gum;
alginates and carrageenans;
glycosaminoglycans, hyaluronic acid and derivatives thereof;
shellac resin, sandarac gum, dammar resins, elemi gums and copal resins;
deoxyribonucleic acid;
mucopolysaccharides such as hyaluronic acid and chondroitin sulfates, and mixtures thereof.

Use will more particularly be made of a thickener chosen from:
copolymers of at least two monomers chosen from acrylic acid, methacrylic acid or an ester thereof of INCI name Acrylates copolymer, in particular the product sold under the name Carbopol Aqua SF1® by the company Lubrizol Advanced Materials, Inc.,
polyacrylic acid/C10-C30 alkyl acrylate crosslinked copolymers of INCI name: Acrylates/C10-C30 alkyl acrylate crosspolymer, such as the products sold under the trade names Pemulen TR1 and Pemulen TR2® or Carbopol 1382®, Carbopol ETD 2020 Polymer®, Carbopol 1342 Polymer®, Carbopol SC 200®, Carbopol SC 500 Polymer®, Carbopol Ultrez 20 Polymer®, Carbopol Ultrez 21 Polymer® from the company Lubrizol Advanced Materials, Inc.;
an acrylamidomethylpropanesulfonic acid polymer partially neutralized with ammonia and crosslinked, of INCI name: Ammonium polyacryloyldimethyl taurate sold under the trade name Hostacerin AMPS® by the company Clariant,
and mixtures thereof.

The thickener(s) may preferably be used in a proportion of from 0.05% to 3% by weight of solids relative to the total weight of the aqueous phase, especially from 0.1% to 2% by weight relative to the total weight of the composition.

### Particulate Dyestuff

The particulate dyestuff is intended in particular to give a coloured appearance or a nacreous effect to compositions that are useful for making up the skin.

The particulate dyestuff is preferably chosen from pigments and nacres, and mixtures thereof.

This dyestuff is preferably present in contents ranging from 5% to 40% by weight and more particularly from 10% to 30% by weight relative to the total weight of the composition. According to a preferred mode of the invention, the dyestuff is present in the composition in a total content ranging from 15% to 25% by weight relative to the total weight of the composition.

### Pigments

The term “pigments” should be understood as meaning white or coloured, mineral or organic particles of any shape, which are insoluble in the medium of the composition, and which are intended to colour the composition.

The pigments may be white or coloured, and mineral and/or organic.

The term “mineral pigment” means any pigment that satisfies the definition in Ullmann's encyclopaedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminium powder or copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The term “organic pigment” means any organic compound which is insoluble in the medium of the composition, and which is intended to colour the composition.

The organic pigments may be chosen from the materials below, and mixtures thereof:
cochineal carmine,
organic pigments of azo dyes, anthraquinone dyes, indigoid dyes, xanthene dyes, pyrene dyes, quinoline dyes, triphenylmethane dyes or fluoran dyes.

Among the organic pigments, mention may be made in particular of the D&C certified pigments known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

The chemical materials corresponding to each of the organic dyestuffs mentioned previously are mentioned in the publication “International Cosmetic Ingredient Dictionary and Handbook”, 1997 edition, pages 371 to 386 and 524 to 528, published by The Cosmetic, Toiletry, and Fragrance Association, the content of which is incorporated into the present patent application by reference.

According to a particular mode, the pigment(s) used may be surface-treated and may especially be coated with at least one hydrophilic compound.

For the purposes of the invention, the “coating” of a pigment according to the invention generally denotes the total or partial surface treatment of the pigment with a surface agent absorbed, adsorbed or grafted onto said pigment.

The surface-treated pigments may be prepared according to surface treatment techniques of chemical, electronic, mechanochemical or mechanical nature that are well known to a person skilled in the art. Commercial products may also be used.

The surface agent may be absorbed, adsorbed or grafted onto the pigments by evaporation of solvent, chemical reaction and creation of a covalent bond.

According to one variant, the surface treatment is constituted of a coating of the pigments.

The coating may represent from 0.1% to 20% by weight and in particular from 0.5% to 10% by weight relative to the total weight of the coated pigment.

The coating may be realized, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be realized, for example, by chemical reaction of a surface agent with the surface of the solid pigment particles and creation of a covalent bond between the surface agent and the particles. This method is notably described in the U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited at the surface of the pigments.

The surface treatment agent may be hydrophilic, such as those described, for example, in Cosmetics & Toiletries, February 1990, Vol. 105, pages 53-64. Mention may be made, for example, of amino acids, C1-C5 alkanolamines, silicon oxides (silica), sodium hexametaphosphate or glycerol or mixtures thereof, PEG-12 dimethicone, sodium glycerophosphate, PEG-7 glyceryl cocoate+methylsilanol tri-PEG-8 glyceryl cocoate+polyquaternium-7, chitosan, methoxy PEG-10 propyltrimethoxysilane, PEG/PPG-18/18 dimethicone, microcrystalline cellulose, and polyethylene glycol alkoxysilanes.

A composition according to the invention may comprise a content of pigments ranging from 0% to 30% by weight relative to the total weight of the composition, preferably ranging from 0% to 20% by weight and preferentially ranging from 1% to 15% by weight, relative to the total weight of the composition. According to a preferred mode of the present invention, the pigments are present in the composition in a content ranging from 1% to 10% by weight relative to the total weight of the composition.

### Nacres

The term “nacre” should be understood as meaning coloured particles of any form, which may or may not be iridescent, especially produced by certain molluscs in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, titanium mica coated especially with an organic dye, and also nacreous pigments based on bismuth oxychloride.

They may also be mica particles, at the surface of which are superposed at least two successive layers of metal oxides and/or of organic dyestuffs.

These particles may be chosen from particles containing a natural or synthetic substrate at least partially coated with at least one layer of at least one metal oxide, chosen, for example, from titanium oxides, especially TiO₂, iron oxides, especially Fe₂O₃, tin oxides, chromium oxides, barium sulfate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures or alloys thereof.

Examples of such particles that may be mentioned include particles including a synthetic mica substrate coated with titanium dioxide, or glass particles, in particular calcium borosilicate particles, coated either with brown iron oxide, with titanium oxide, with tin oxide or with a mixture thereof, for instance the products sold under the brand name Reflecks® by the company Engelhard.

The nacres may have a yellow, pink, red, bronze, orange, brown, gold and/or coppery tint.

Non-limiting examples of nacres that may be mentioned in particular, alone or as a mixture, include nacres including the following elements:
mica-titanium oxide (for example sold under the commercial reference Timica Sparkle 110RD from BASF, Flamenco Blue® from BASF, Candurin Brown Amber® from Merck, Pearlescent Pigment Prestige Gold® from Sudarshan);
mica-titanium oxide coated with N-lauroyl-L-lysine (for example sold under the commercial reference LLD-10 Flamenco Violet® from Daito Kasei Kogyo);
mica-iron oxide (for example sold under the commercial reference Pearlescent Pigment Prestige Soft Bronze® from Sudarshan, Colorona Bronze Sparkle® from Merck, Timica Golden Bronze® from BASF, Candurin Light Gold® from Merck, Duocrome YR 422 C® from BASF, Gemtone Tan Opal G 005® from BASF);
mica-titanium oxide-iron oxide (for example sold under the commercial reference Timica Brilliant Gold 212 G Gemtone Goldstone G0014® or Timica Golden Bronze® from BASF);
mica-bismuth oxychloride-iron oxide (for example sold under the commercial reference Chroma-Lite Yellow® from BASF);
mica-titanium oxide-chromium oxide-iron oxide (for example sold under the commercial reference Gemtone Jade® from BASF);
mica-titanium oxide-tin oxide (for example sold under the commercial reference Flamenco Super Blue 630 Z® from BASF, Helios R100R® from Topy);
mica-silica-titanium oxide (for example sold under the commercial reference Timiron Splendid Red® from Merck);
platelets of sodium calcium borosilicate coated with titanium oxide and tin oxide (for example sold under the commercial reference Reflecks Rays of Red G430L® from BASF);
platelets of sodium calcium borosilicate coated with tin oxide and iron oxide (for example sold under the commercial reference Mirage Sparkling Champagne® from Eckart);
platelets of calcium aluminium borosilicate coated with titanium oxide and tin oxide, optionally treated with silica (for example sold under the commercial reference Ronastar® from Merck);
platelets of calcium aluminium borosilicate coated with silica oxide and tin oxide (for example sold under the commercial reference Ronastar Golden Jewel SQ® from Merck);
glass powder coated with titanium oxide (for example sold under the commercial reference Reflecks Dimensions Glittering Gold G2305® from BASF or Metashine MC112ORY® from NSG);
alumina and titanium oxide (for example sold under the commercial reference Spectraflex Focus Red C88-1031® from Sun);
bronze powder (for example sold under the commercial reference Visionaire Bright Sunflower Gold® from Eckart);
synthetic fluorophlogopite and magnesium silicate and titanium dioxide and tin oxide (for example sold under the commercial reference Sunshine Daybreak Blue® from Sun Chemical).

For the purposes of the present invention, the term “mica” includes natural micas and synthetic micas.

According to a particularly preferred mode, the particulate dyestuff will be chosen from titanium dioxide particles, iron oxide particles, and mixtures thereof.

According to a particularly preferred form of the invention, the composition in the form of an aqueous particle dispersion especially comprising a physiologically acceptable medium, especially for coating keratin materials, more particularly for making up and/or caring for keratin materials, such as the skin, contains:

a) an aqueous phase; and
b) at least one matt-effect filler chosen from boron nitride, polymethylsilsesquioxane powders, and mixtures thereof;
c) as solid particles formed from a film-forming polymer suspended in the aqueous phase, at least one aqueous dispersion of aminomethylpropanol salt of a copolymer of allyl methacrylate and of one or more monomers chosen from acrylic acid, methacrylic acid or an ester thereof of INCI name: AMP-acrylates/allyl methacrylate copolymer; and
d) at least one ionic polymeric dispersant chosen from homopolymers of the sodium salt of methacrylic acid;
e) at least one thickener chosen from:
copolymers of at least two monomers chosen from acrylic acid, methacrylic acid or an ester thereof;
polyacrylic acid/C₁₀-C₃₀ alkyl acrylate crosslinked copolymers;
an acrylamidomethylpropanesulfonic acid polymer partially neutralized with ammonia and crosslinked; and mixtures thereof, and
   f) at least one particulate dyestuff chosen from titanium dioxides, iron oxides, and mixtures thereof.

### Additives

The compositions according to the invention may also comprise additional cosmetic ingredients conventionally used in aqueous cosmetic compositions especially for making up and/or caring for the skin. Examples that may especially be mentioned include additives chosen from polyols, monoalcohols, water-soluble dyestuffs, preserving agents, antioxidants, chelating agents, sequestrants, antifoams, fragrances, sunscreens, bactericidal agents, water-soluble active agents, and mixtures thereof.

Needless to say, a person skilled in the art will take care to select the optional additional ingredients and/or the amount thereof such that the advantageous properties of the composition according to the invention are not, or are not substantially, adversely affected by the envisaged addition.

More particularly, the compositions according to the invention contain at least one additive chosen from polyols, monoalcohols, and mixtures thereof.

### Polyol

The composition may also comprise a polyol that is miscible with water at room temperature (25° C.) chosen especially from polyols containing from 2 to 20 carbon atoms, preferably containing from 2 to 10 carbon atoms and preferentially containing from 2 to 8 carbon atoms, such as glycerol, propylene glycol, 1,3-butylene glycol, pentylene glycol, hexylene glycol, caprylyl glycol, dipropylene glycol or diethylene glycol; glycol ethers (especially containing from 3 to 16 carbon atoms) such as mono-, di- or tripropylene glycol (C₁-C₄)alkyl ethers, mono-, di- or triethylene glycol (C₁-C₄)alkyl ethers; polyethylene glycols; and mixtures thereof.

Use will more preferentially be made of propylene glycol.

According to a preferential mode of the invention, the polyol(s) are preferably present in a content ranging from 1% to 20% by weight relative to the total weight of the composition, and more particularly ranging from 5% to 15% by weight relative to the total weight of the composition.

### Monoalcohol

In addition, the composition according to the invention may comprise a monoalcohol containing from 2 to 5 carbon atoms, such as ethanol or isopropanol.

Ethanol will be used more preferentially.

According to a preferential mode of the invention, the monoalcohol is present in a content ranging from 1% to 10% by weight relative to the total weight of the composition, and preferably in a content ranging from 3% to 7% by weight relative to the total weight of the composition.

### Additional Dyestuffs

According to a particularly preferred mode of the invention, the compositions also contain at least one water-soluble dyestuff.

For the purposes of the invention, the term “water-soluble dyestuff” means any natural or synthetic, generally organic compound, which is soluble in an aqueous phase or water-miscible solvents and which is capable of colouring.

As water-soluble dyes that are suitable for use in the invention, mention may be made especially of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophyllin, methylene blue, anthocyanins (enocianin, black carrot, hibiscus and elder), caramel and riboflavin.

### Cosmetic Applications

According to one embodiment, a composition of the invention may advantageously be in the form of a composition for caring for the skin of the body or the face, in particular the face.

According to another embodiment, a composition of the invention may advantageously be in the form of a composition for making up the skin of the body or the face, in particular the face.

A composition of the invention may advantageously be in the form of a foundation.

According to another mode of this embodiment, a composition of the invention may advantageously be in the form of an eyeshadow or a face powder.

Such compositions are especially prepared according to the general knowledge of those skilled in the art.

### Cosmetic Kit

According to another aspect, the invention also relates to a cosmetic assembly comprising:
i) a container delimiting at least one compartment, said container being closed by a closing member; and
ii) a composition as described previously, placed inside said compartment.

The container may be in any suitable form. It may especially be in the form of a bottle, a tube, a jar, a case, a can, a sachet or a box.

The closing member may be in the form of a removable stopper, a lid, a cap, a tear-off strip or a capsule, especially of the type including a body attached to the container and a cover cap articulated on the body. It may also be in the form of a member for selectively closing the container, especially a pump, a valve or a flap valve.

The container may be combined with an applicator. The applicator may be in the form of a fine brush, as described, for example, in patent FR 2 722 380. The applicator may be in the form of a foam or elastomer pad, of a felt-tipped pen or of a spatula. The applicator may be free (powder puff or sponge) or securely fastened to a stem borne by the closing member, as described, for example, in patent U.S. Pat. No. 5,492,426. The applicator may be solidly attached to the container, as described, for example, in patent FR 2 761 959.

The product may be contained directly in the container, or indirectly. By way of example, the product may be arranged on an impregnated support, especially in the form of a wipe or a pad, arranged (individually or in plurality) in a box or in a sachet. Such a support incorporating the product is described, for example, in patent application WO 01/03538.

The closing member may be coupled to the container by screwing. Alternatively, the coupling between the closing member and the container is done other than by screwing, especially via a bayonet mechanism, by click-fastening, clamping, welding, adhesive bonding or by magnetic attraction. The term “click-fastening” is understood to mean, in particular, any system that involves surmounting a rim or bead of material by elastic deformation of a portion, especially of the closing member, then by returning to the elastically unstressed position of said portion after the rim or bead has been surmounted.

The container may be at least partially made of thermoplastic material. Examples of thermoplastic materials that may be mentioned include polypropylene and polyethylene.

Throughout the description, including the claims, the term “including a” should be understood as being synonymous with “including at least one”, unless otherwise specified.

The expressions “between . . . and . . . ”, and “ranging from . . . to . . . ” should be understood as meaning limits included, unless otherwise specified.

The invention is illustrated in greater detail by the examples and figures presented below. Unless otherwise indicated, the amounts shown are expressed as weight percentages.

### EXAMPLE

### Example 1: Foundation in the Form of an Aqueous Particle Dispersion

| | | Concentration |
|---|---|---|
| Phase | INCI name | in % by weight |
| A1 | Water | 25.00 |
| | Acrylates/C10-C30 Alkyl Acrylate Crosspolymer | 0.07 |
| | (Carbopol 1342 Polymer ®) | |
| | Ammonium Polyacryloyldimethyl Taurate | 1.00 |
| | (Hostacerin AMPS ®) | |
| | Triethanolamine | 0.13 |
| | Sodium polymethacrylate (Darvan-7N ®) | 0.95 |
| | Propylene glycol | 8.00 |
| | Synthetic fluorophlogopite | 1.43 |
| | Titanium dioxide (CI: 77891) | 8.90 |
| | Yellow iron oxides (CI: 77492) | 1.21 |
| | Red iron oxides (CI: 77491) | 0.26 |
| | Black iron oxides (CI: 77499 ) | 0.12 |
| A2 | Water | qs 100 |
| | AMP-Acrylates/allyl methacrylate copolymer | 5.00 |
| | (Fixate G-100L PR Polymer ®) as an aqueous | |
| | dispersion at 25-28% by weight | |
| | Phenoxyethanol | 0.64 |
| | Disodium EDTA | 0.5 |
| | Chlorphenesin | 0.25 |
| | Dimethicone (and) polysorbate 65 (and) | 0.15 |
| | simethicone | |
| | Acrylates copolymer | 0.5 |
| | (Carbopol Aqua SF-1 Polymer ®) | |
| B | Polymethylsilsesquioxane (Tospearl 145A ®) | 2.00 |
| | Boron nitride (Boron Nitride SHP 3 ®) | 1.00 |
| C | Denat. alcohol | 5.00 |

Procedure:

The composition was manufactured at room temperature (25° C.) in the following manner.

Phase A1 was prepared by introducing the two thickening polymers into the water in a tank with stirring using a paddle mixer for 15 minutes at 4000 rpm, then the triethanolamine was added with stirring for 5 minutes at the same speed. The propylene glycol and the sodium polymethacrylate were successively added with stirring for 5 minutes at the same speed, then the pigments and the synthetic mica were added at the same speed. After one minute, the walls were scraped and turbomixing was performed for 20 minutes at the same speed. Phase A2 was prepared by introducing the various ingredients into the water, which was added to the tank containing phase A1. Phase B and phase C were then successively added to the resulting mixture with stirring for 20 minutes at the same speed.

An aqueous dispersion in which the pigments are uniformly dispersed was obtained.

### Tests of the Matt Effect and the Persistence of the Matt Effect

### Measurement Principle

The gloss of the skin is measured using the Samba FDT_Fr2 polarimetric camera, which is a black and white polarimetric imaging system, with which images are acquired in parallel (P) and crossed (C) polarized light. By analysing the image resulting from subtraction of the two images (P-C), the gloss is quantified, by measuring the average greyscale of the brightest 5% of pixels corresponding to the gloss areas.

### Test Procedure

The test proceeds in the following manner:

The test is performed in vivo on 17 Caucasian individuals from 18 to 65 years old, with greasy, sparingly to moderately wrinkled skin, a pale flesh tone, and toned skin with no wrinkles or redness on the cheeks. Each volunteer receives two products for half-face testing. This test is performed in parallel groups and the attribution of the individuals to the treatment groups is randomized.

The participants arrive in an air-conditioned waiting room (22° C.±2° C.) 15 minutes before the start of the test. They remove their makeup and an image of one of their cheeks is taken with the polarimetric camera. This image allows measurement of the gloss at T0 before applying makeup.

Next, 100 mg of foundation are weighed out on a watch glass, and are applied with the bare fingers to the half-face on which the measurement at T0 was taken.

After a drying time of 15 minutes, an image of the made-up cheek is taken with the polarimetric camera. This image allows measurement of the gloss just after applying makeup (Timm).

The models then return to the air-conditioned room for 3 hours.

Finally, an image of the made-up cheek after the waiting time of 3 hours is taken with the polarimetric camera. This image allows measurement of the gloss after 3 hours of makeup (T3h).

### Expressing the Results

The difference ΔE (Timm−T0) which measures the effect of the makeup on the skin is calculated. A negative value means that the makeup reduces the gloss of the skin and that it thus has a matt effect.

The difference ΔE (T3h−Timm) which measures the persistence of this effect is then calculated. The value obtained should be as low as possible, which means that the makeup effect does not change over time.

The results obtained are shown in the table below.

| | Immediate matt | Matt effect |
|---|---|---|
| | effect ΔE | persistence ΔE |
| | (Timm − To) | (T3 h − Timm) |
| Matt effect of | −6.43 ± 0.95 | 1.25 ± 0.88 |
| Example 1 | | |

The composition according to the invention had a good matt effect and good persistence of this effect after 3 hours.

### Tests of Colour and of Matt Effect Persistence

### Measurement Principle

The colouring of the skin is measured using a skin image acquisition device such as the Chromasphere® machine as described in patent EP1288706 and according to the method indicated in said document.

### Test Procedure

The test proceeds in the following manner:

The test is performed in vivo on 17 Caucasian individuals from 18 to 65 years old, with greasy, sparingly to moderately wrinkled skin, a pale flesh tone, and toned skin with no wrinkles or redness on the cheeks. Each volunteer receives two products for half-face testing. This test is performed in parallel groups and the attribution of the individuals to the treatment groups is randomized.

The participants arrive in an air-conditioned waiting room (22° C.±2° C.) 15 minutes before the start of the test. They remove their makeup and the colour of one of their cheeks at T0 before being made up is evaluated using the Chromasphere® machine.

Next, 100 mg of foundation are weighed out on a watch glass, and are applied with the bare fingers to the half-face on which the measurement at T0 was taken.

After a drying time of 15 minutes, the immediate colouring of the cheek after application (Timm) is measured.

The models then return to the air-conditioned room for 3 hours.

The immediate colouring of the skin after application of the made-up cheek is measured after a waiting period of 3 hours using the Chromasphere® machine.

This image allows measurement of the colouring of the made-up cheek after 3 hours of makeup (T3h).

### Expressing the Results

The difference ΔE (Timm−T0) which measures the effect of the makeup on the skin is calculated.

The difference ΔE (T3h−Timm) which measures the persistence of this colour effect is then calculated. The value obtained should be as low as possible, which means that the colour effect does not change over time.

The results obtained are shown in the table below.

| | Immediate colouring | Persistence of |
|---|---|---|
| | effect ΔE | the colouring ΔE |
| | (Timm − To) | (T3 h − Timm) |
| Colouring effect | 3.60 ± 0.46 | 0.64 ± 0.11 |
| of Example 1 | | |

The composition according to the invention had a good colouring effect and good persistence of this effect after 3 hours.

### Comparative Sensory Test of Transfer-Resistance Effect

The transfer-resistance effect of the composition of Example 1 according to the invention in the form of an aqueous coloured particle dispersion was compared with that of two reference products on the market of water-based foundations in the form of a water-in-oil emulsion, namely:

1) the product WATER BLEND FACE & BODY FOUNDATION® (i.e.: Mintel product No. 4792105) the ingredient list of which is:
WATER, CI 77891, HYDROGENATED POLYISOBUTENE, SORBITAN TRIOLEATE, PENTAERYTHRITYL TETRAISOSTEARATE, METHYLPROPANEDIOL, GLYCERIN, FRAGRANCE, MANGIFERA INDICA SEED BUTTER, BUTYLENE GLYCOL, PHENOXYETHANOL, GLYCERYL CAPRYLATE, TROMETHAMINE, CI 77492, ACRYLATES/C10-30 ALKYL ACRYLATE CROSSPOLYMER, 1,2-PENTANEDIOL, PANTHENOL, ALUMINUM DIMYRISTATE, CI 77491, CI 77499, BUTYLATED HYDROXYTOLUENE, TOCOPHEROL (VITAMIN E);
2) the product LIQUID AIR TOO COOL FOR SCHOOL ART CLASS® (i.e.: Mintel product No. 4891985) in the form of a water-in-oil emulsion, the ingredient list of which is:
WATER, PHENYL TRIMETHICONE, ALCOHOL DENAT., DIMETHICONE, ISODODECANE, ETHYLHEXYL METHOXYCINNAMATE, SILICA, BUTYLENE GLYCOL, PEG-10 DIMETHICONE, TITANIUM DIOXIDE, METHYL METHACRYLATE CROSSPOLYMER, POLYMETHYL SILSESQUIOXANE, MICA, SODIUM CHLORIDE, DIMETHICONE/PEG-10/15 CROSSPOLYMER, ISOHEXADECANE, SQUALANE, POTASSIUM SORBATE, SODIUM DEHYDROACETATE, DISODIUM STEAROYL GLUTAMATE, TOCOPHERYL ACETATE, FRAGRANCE, ISOPROPYL TITANIUM TRIISOSTEARATE, MALTODEXTRIN, DRUMSTICK SEED EXTRACT, XYLITYL GLUCOSIDE, ALUMINIUM HYDROXIDE, ANHYDROXYLITOL, XYLITOL, DIPROPYLENE GLYCOL, CITRONELLOL, BENZYL BENZOATE, BUTYLPHENYL METHYLPROPIONAL, LINALOOL, SODIUM CITRATE, ALPHA-ISOMETHYL IONONE, COUMARIN, TOCOPHEROL, TITANIUM DIOXIDE, YELLOW IRON OXIDE, RED IRON OXIDE, BLACK IRON OXIDE.

A sensory test was performed on a panel of 14 women with normal skin. Each evaluating person, skilled in working with foundations, was trained by a reference foundations expert regarding the score attributed to reference products for the transfer-resistance criterion. The participants are foundation users who show great interest in cosmetic products. Their memory, rigour, mathematical logic, concentration and motivational capacities enabled them to meet the conditions for gaining access to the status of participants on conclusion of a training course. The protocol used is an in vivo protocol that is as close as possible to the gestures used by consumers, thus representing a natural gesture for personally applying a foundation formulation. The transfer-resistance criterion was evaluated on a scoring scale from 0 to 5. The higher the value, the greater the transfer.

The results obtained are indicated in the table below with the mean of the scores obtained:

| | | | LIQUID AIR |
|---|---|---|---|
| | | WATER BLEND | TOO COOL |
| | | FACE & BODY | FOR SCHOOL |
| | Example 1 | FOUNDATION ® | ART CLASS ® |
| Composition | (invention) | (reference) | (reference) |
| Transfer- | 2.7 | 3.9 | 4.2 |
| resistance | | | |
| effect | | | |

Composition 1 according to the invention in the form of an aqueous coloured particle dispersion produced a significantly better transfer-resistance effect than the reference aqueous formulations on the market of foundations in water-in-oil emulsion form.

1. A composition in the form of an aqueous particle dispersion, comprising a physiologically acceptable medium, for coating keratin materials, containing:
a) an aqueous phase;
b) at least one matt-effect filler;
c) solid particles formed from at least one film-forming polymer suspended in said aqueous phase;
d) at least one ionic polymeric dispersant;
e) at least one thickener; and
f) at least one particulate dyestuff.

2. The composition according to claim 1, in which the matt-effect filler(s) are present in concentrations ranging from 0.1% to 5% by weight relative to the total weight of the composition.

3. The composition according to claim 1 wherein the filler(s) are selected from the group consisting of silica powders; polytetrafluoroethylene powders; silicone resin powders; acrylic copolymer powders; hollow hemispherical silicone particles; hydrophobic silica aerogel particles; crosslinked elastomeric organopolysiloxane powders; crosslinked elastomeric organopolysiloxane powders coated with silicone resin; polyamide powders; starch powders; talc/TiO₂/alumina/silica composite powders; silica/TiO₂ composites; spherical cellulose particles; clays; perlite; boron nitride, and mixtures thereof.

4. The composition according to claim 1, wherein the filler(s) are selected from the group consisting of:
silica powders;
polymethylsilsesquioxane powders;
polymethyl (meth)acrylate (PMMA) powders;
perlite;
boron nitride;
and mixtures thereof.

5. The composition according claim 1, wherein the film-forming polymer(s) are present in a solids content ranging from 0.1% to 5% by weight relative to the total weight of the composition.

6. The composition according to claim 1, wherein the film-forming polymer is selected from the group consisting of:
aqueous dispersions of acrylic polymer;
aqueous dispersions of polyurethane;
aqueous dispersions of sulfopolyester;
vinyl dispersions;
aqueous dispersions of polyurethane/polyacrylic hybrid polymer;
aqueous dispersions of particles of core-shell type; and
mixtures thereof.

7. The composition according to claim 1, wherein the film-forming polymer is an aqueous dispersion of aminomethylpropanol salt of a copolymer of allyl methacrylate and of one or more monomers chosen from acrylic acid, methacrylic acid or an ester thereof of INCI name: AMP-acrylates/allyl methacrylate copolymer.

8. The composition according to claim 1, wherein the ionic polymeric dispersant is selected from the group consisting of:
(meth)acrylic acid homopolymers and salts thereof,
(meth)acrylic acid copolymers and salts thereof,
styrene/(meth)acrylic acid copolymers and salts thereof,
vinylnaphthalene/acrylic acid copolymers and salts thereof,
styrene/maleic acid copolymers and salts thereof,
vinylnaphthalene/maleic acid copolymers and salts thereof,
copolymers of maleic anhydride and of diisobutylene, and salts thereof, and
mixtures thereof.

9. The composition according to claim 1, wherein the ionic polymeric dispersant is selected from the group consisting of:
salts of copolymers of at least two or more monomers chosen from the group comprising acrylic acid, methacrylic acid or an ester thereof,
salts of styrene/maleic anhydride copolymer,
salts of methacrylic acid homopolymer,
and mixtures thereof, and more particularly a homopolymer of the sodium salt of methacrylic acid, especially as an aqueous 25% solution.

10. The composition according to claim 1, including a total content of ionic polymeric dispersant(s) of from 0.05% by weight to 3% by weight relative to the total weight of the composition.

11. The composition according to claim 1, wherein the thickener is selected from the group consisting of:
copolymers of at least two monomers chosen from acrylic acid, methacrylic acid or an ester thereof;
polyacrylic acid/C10-C30 alkyl acrylate crosslinked copolymers of INCI name:
Acrylates/C10-C30 Alkyl Acrylate Crosspolymer;
an acrylamidomethylpropanesulfonic acid polymer partially neutralized with ammonia and crosslinked, of INCI name: Ammonium Polyacryloyldimethyltaurate;
and mixtures thereof.

12. The composition according to claim 1, wherein the thickener(s) are present in a proportion of from 0.05% to 3% by weight of solids relative to the total weight of the aqueous phase and from 0.1% to 2% by weight relative to the total weight of the composition.

13. The composition according to claim 1, wherein the particulate dyestuff is selected from the group consisting of pigments, nacres, and mixtures thereof.

14. The composition according to claim 1, wherein the particulate dyestuff(s) are present in contents ranging from 5% to 40% by weight, relative to the total weight of the composition.

15. Composition according to claim 1, wherein the particulate dyestuff(s) are selected from the group consisting of titanium dioxide particles, iron oxide particles, and mixtures thereof.

16. The composition according to claim 1, wherein the aqueous phase is present in a content ranging from 70% to 95% by weight relative to the total composition.

17. The composition according to claim 1, wherein water is present in a content ranging from 40% to 80% by weight relative to the total composition.

18. The composition according to claim 1, containing further comprising at least one additive selected from polyols, monoalcohols, and mixtures thereof.

19. The composition according to claim 1, which is a foundation.

20. The composition according to claim 1 which is the form of an eyeshadow or a face powder.

21. A process for coating keratin materials, comprising: applying to the keratin materials a composition according to claim 1.
